(19) United States
(12) Patent Application Publication
(10) Pub. No.: US 2009/0258836 A1
(43) Pub. Date: Oct. 15, 2009

(54) EFFECTIVE DELIVERY OF CROSS-SPECIES A3 ADENOSINE-RECEPTOR ANTAGONISTS TO REDUCE INTRAOCULAR PRESSURE

(75) Inventors: Mortimer M. Civan, Philadelphia, PA (US); Kenneth A. Jacobson, Silver Spring, MD (US); Marcel Y. Avila, Bogota (CO); Richard Stone, Havertown, PA (US)

Correspondence Address: MONTGOMERY, MCCRACKEN, WALKER & RHOADS, LLP, 123 SOUTH BROAD STREET, AVENUE OF THE ARTS, PHILADELPHIA, PA, 19109, US

(73) Assignee: The Trustees of The University of Pennsylvania

(21) Appl. No.: 12/419,062

(22) Filed: Apr. 6, 2009

Related U.S. Application Data
(63) Continuation of application No. PCT/US2007/021409, filed on Oct. 5, 2007.
(60) Provisional application No. 60/850,175, filed on Oct. 6, 2006.

(51) Int. Cl. A61K 31/7076; A61P 27/06

(52) U.S. Cl. 514/46; 514/45

(57) ABSTRACT

Provided are methods for reducing intraocular pressure in an individual having an ocular disorder causing elevated intraocular pressure, such as glaucoma. The method comprises administering to the individual an effective intraocular pressure-reducing amount of a pharmaceutical composition comprising an A₃ subtype adenosine receptor (A₃AR) antagonist, including dihydropyridine, pyridine, pyridinium salt or triazoloquinazoline, and derivatives thereof expressly having A₃AR antagonist activity, including, e.g., the nucleoside-based A₃AR antagonist, MRS-3820. Further provided is a method for ensuring the delivery of a topically administered therapeutic composition for reducing intraocular pressure, wherein the method expressly requires physically opening a channel through the corneal barrier of the patient's eye by a microneedle or micropipette to permit transport of the topical composition to the anterior chamber of the eye.

### CROSS-REFERENCE TO RELATED APPLICATIONS

This application is a continuation of International Application PCT/US2007/021409 filed on Oct. 5, 2007 and published on Apr. 17, 2008, which claims priority to U.S. Provisional Application 60/850,175 filed on Oct. 6, 2006, each of which is incorporated herein in its entirety.

### GOVERNMENT INTEREST

This invention was supported in part by Grant Nos. EY08343 and EY013624 from the U.S. National Institutes of Health. The U.S. Government may therefore have certain rights in this invention.

### FIELD OF THE INVENTION

The present invention relates to the use of A₃ subtype adenosine receptor antagonists as a cross-species pharmaceutical for reducing intraocular pressure, and methods for assuring effective delivery to the target site.

### BACKGROUND OF THE INVENTION

Glaucoma, a disorder characterized by increased intraocular pressure (IOP), is a leading cause of irreversible blindness (Quigley et al., Br. J. Opthalmol. 80:389-393 (1996)). Intraocular pressure is determined by the rate of inflow of aqueous humor across the ciliary epithelium and the resistance to outflow from the anterior chamber of the eye. At fixed outflow resistance, an increase in inflow will increase IOP until the sum of the pressure-dependent and pressure-independent outflows matches inflow. Increased IOP typically leads to retinal ganglion cell death and optical nerve atrophy. Reducing the elevated IOP is the only strategy that is, to date, unequivalently documented as a method for delaying the onset of, and slowing the progression of, glaucomatous blindness. Many transport components underlying inflow are known (FIG. 1), but their regulation is poorly understood.

The aqueous humor of the eye is formed by the ciliary epithelium, which comprises two cell layers: the outer pigmented epithelial (PE) cells facing the stroma and the inner non-pigmented epithelial (NPE) cells in contact with the aqueous humor. The activity of Cl⁻ channels is likely to be a rate-limiting factor in aqueous humor secretion, given the low baseline level of channel activity and the predominance of the chloride anion in the fluid transferred (Coca-Prados et al., Am. J. Physiol. 268: C572-C579 (1995)). Thus, the secretion of aqueous humor into the eye is believed to be a consequence of two opposing physiological processes: fluid secretion into the eye by the NPE cells and fluid reabsorption (secretion out of the eye) by the PE cells. Further, the release of chloride ion (Cl⁻) by the non-pigmented ciliary epithelial (NPE) cells into the adjacent aqueous humor via Cl⁻ channels appears to enhance secretion, whereas Cl⁻ release by the pigmented ciliary epithelial (PE) cells into the neighboring stroma appears to reduce net secretion (Civan, Current Topics in Membranes 45:1-24 (1998); Civan, News Physiol. Sci. 12:158-162 (1997)). Adenosine has been found to activate NPE Cl⁻ channels, which enables Cl⁻ release (Carre et al., Am. J. Physiol. (Cell Physiol. 42) 273:C1354-C1361 (1997)). Purines, a class of chemical compounds which includes adenosine, ATP and related compounds, may regulate aqueous humor secretion, in part through modification of the Cl⁻ channel activity. Both NPE and PE cells have been reported to release ATP to the extracellular surface, where ATP can be metabolized to adenosine by ecto-enzymes (Mitchell et al. Proc. Natl. Acad. Sci. U.S.A. 95:7174-7178 (1998)), and both cell types possess adenosine receptors (Wax et al., Exp. Eye Res. 57:89-95 (1993); Wax et al, Invest. Opthalmol. Vis. Sci., 35:3057-3063 (1994); Kvanta et al., Exp. Eye Res. 65:595-602 (1997)) and ATP receptors (Wax et al., supra, 1993; Shahidullah et al., Curr. Eye Res. 16:1006-1016 (1997)). Furthermore, in vitro studies of rabbits have associated A₂-adenosine receptors with increased secretion and elevated intraocular pressure (Crosson et al., Invest. Opthalmol. Vis. Sci. 37:1833-1839 (1996)) and A₁-adenosine receptors with the converse (Crosson, J. Pharmacol. Exp. Ther. 273:320-326 (1995)). Qualitatively similar associations with intraocular pressure, but not with secretion, have been observed in cynomologus monkeys (Tain et al., Exp. Eye Res., 64:979-989 (1997)).

Intraocular pressure has also been reduced by stimulating reabsorption of aqueous humor. In principle, this could be achieved by activating chloride channels on the basolateral surface of the pigmented cell layer, which would release chloride back into the stroma. In PE cells, this has been accomplished using the antiestrogen, tamoxifen.

Alternatively, adenosine receptors (ARs) have been a promising target for lowering IOP. This is because knockout of A₃-adenosine receptors has been shown to reduce IOP in vivo in the mouse. In vitro observations indicate that the knockout triggered reduction in IOP is mediated through a reduction in the inflow. When combined, published reports have shown that: 1) adenosine activates NPE-cell Cl⁻ channels (Carré et al., supra, 1997); 2) the Cl⁻ channel activation is mediated by A₃ARs (Mitchell et al., Am. J. Physiol. 276:C659-C666 (1999)); 3) the A₃AR-activated Cl⁻ channels constitute a major fraction of the total NPE-cell Cl⁻ channels (Carrè et al., Am. J. Physiol.: Cell Physiol. 279:C440-C451 (2000)); 4) A₃AR antagonists lower IOP of the mouse eye (Avila et al., Br. J. Pharmacol. 134:241-245 (2001); Avila et al., Invest. Opthalmol. Vis. Sci. 43:3021-3026 (2002); Yang et al., Curr. Eye Res. 30:747-754 (2005)); and 5) IOP of A₃ subtype adenosine receptor (A₃AR)-null mice is unresponsive to the A₃AR-antagonist MRS-1191. Specifically, A₃AR agonists reportedly increase or stimulate Cl⁻ channels in immortalized human and freshly-dissected bovine NPE cells and of aqueous-oriented Cl⁻ channels of the intact rabbit iris-ciliary body, while A₃AR antagonists lower Cl⁻ channel activity of the NPE cells facing the aqueous surface of the ciliary epithelium (Carre et al., supra, 1997; Mitchell et al., supra, 1999). In contrast, A₃AR agonists exert relatively little effect on cells from conventional outflow pathways (Fleischhauser et al., J. Membr. Biol. 193:121-136 (2003); Karl et al. Am. J. Physiol. Cell Physiol. 288:C784-C794 (2005)).

Current drugs prescribed for glaucoma, in the form of eyedrops, include pilocarpine, timolol, betaxolol, levobunolol, metipranolol, epinephrine, dipivefrin, latanoprost, carbachol, and potent cholinesterase inhibitors, such as echothiophate, and carbonic anhydrase inhibitors, such as dorzolamidet. Many of these effective approaches to medical therapy of glaucoma involve a reduction in the rate of flow into the eye. However, to date, none of these drugs have been satisfactory, in part due to side effects and inconvenient dosing schedules, and cross-species effectiveness has not been previously reported. Nevertheless, there has remained an ongoing need to confirm that A₃AR antagonist compounds are useful cross-species for reducing IOP for the treatment of glaucoma, with improved efficacy, prolonged action and reduced side effects; and also to determine if certain modes of administering therapeutic pharmaceutical compounds to the eye are more effective than others.

### SUMMARY OF THE INVENTION

The present invention addresses the need for compounds capable of reducing intraocular pressure for the treatment of glaucoma with improved efficacy, prolonged action and reduced side effects, and further shows the delivery of a species-independent potent A₃ inhibitor across the cornea, thus avoiding substantial species variation in the response of A₃ subtype adenosine receptors to antagonists. It is important to demonstrate that a favorable response in a laboratory rodent is also representative of a similarly favorable effect in humans. This is particularly important since the mouse is a favored laboratory animal for studying the functional implications of spontaneous and bioengineered mutations. Therefore, in one embodiment of the present invention, the preferred methods for reducing intraocular pressure in the eye, comprise a step of administering to the subject animal or patient an effective intraocular pressure-reducing amount of a cross-species pharmaceutical composition comprising an A₃ subtype adenosine receptor antagonist. In one aspect of this embodiment, the A₃ receptor antagonist is a dihydropyridine, pyridine, pyridinium salt or triazoloquinazoline. Derivatives of compounds selected from these classes, expressly having A₃ receptor antagonist activity, are further contemplated within the present invention.

In an express embodiment, the A₃ subtype receptor antagonist may be selected from among MRS-1097, MRS-1191, MRS-1220, MRS-1523, MRS-1292, MRS-1523, MRS-3642, MRS-3771, MRS-3826, MRS-3827, MRS-3820, MRS 1220, LJ-1830, LJ-1831, LJ-1833, LJ-1834, LJ-1835, LJ-1836, and LJ-1837. Application of an exemplary drug (MRS-3820), which is a nucleoside-based, cross-species, A3-subtype adenosine-receptor (AR) antagonist, is described below for the general purposes of the present invention to lower intraocular pressure (IOP) in vivo, providing a therapeutic effect for glaucomatous patients.

Advantageously, the pharmaceutical composition is administered topically, systemically or orally. Preferably, the pharmaceutical composition is an ointment, gel, eye drops or injectable. It is an object, therefore, to determine whether the cornea presents a substantial barrier to the therapeutic delivery of such pharmaceutical compositions to the interior of the eye by topical application of drops to the tear film.

Additional objects, advantages and novel features of the invention will be set forth in part in the description, examples and figures which follow, all of which are intended to be for illustrative purposes only, and not intended in any way to limit the invention, and in part will become apparent to those skilled in the art on examination of the following, or may be learned by practice of the invention.

### BRIEF DESCRIPTION OF THE FIGURES

The foregoing summary, as well as the following detailed description of the invention, will be better understood when read in conjunction with the appended drawings. It should be understood, however, that the invention is not limited to the precise arrangements and instrumentalities shown.

FIG. 1 is a schematic diagram showing the ocular non-pigmented epithelial (NPE) and pigmented epithelial (PE) cells, and the effects of ATP, adenosine (Ado) and tamoxifen (TMX) on the movement of aqueous humor. Ecto=ecto-enzymes; A₃=A₃ subtype adenosine receptor.

FIGS. 2A-2B show the effect of A₃ antagonists on the IB-MECA-stimulated isotonic shrinkage of NPE cells. FIG. 2A shows that the A₃-selective antagonist MRS-1097 (300 nM) prevented shrinkage triggered by the A₃-selective agonist N⁶-(3-iodobenzyl)-adenosine-5′-N-methyluronamide (IB-MECA) (P<0.01, F-distribution). FIG. 2B shows that the A₃-selective antagonist MRS-1191 (100 nM) prevented characteristic shrinkage triggered by IB-MECA (n=4, P<0.01 by F-distribution). MRS-1191 did not affect cell volume in the absence of IB-MECA, confirming the specificity of the interaction (n=4). Solid trajectories are least-square fits with monoexponentials, whereas data sets displaying no significant shrinkage are connected by dotted lines.

FIGS. 3A-3C show the effects of selective A₃-receptor antagonists on adenosine-stimulated isotonic shrinkage of NPE cells. Application of 300 nM MRS-1097 (FIG. 3A; n=4), 100 nM MRS-1191 (FIG. 3B; n=3), and 100 nM MRS-1523 (FIG. 3C; n=3) all prevented the characteristic shrinkage triggered by nonselective activation of adenosine receptors with 10 μM adenosine (P<0.01, F-distribution). Solid trajectories are least-square fits with mono-exponentials, whereas data sets displaying no significant shrinkage are connected by dotted lines.

FIGS. 4A-4C show the effects of adenosine-receptor agonists on isosmotic volume of NPE cells. In FIG. 4A, the A₃-selective agonist IB-MECA produced prompt shrinkage at 100 nM (n=4, v_{∞}=95.6±0.2%, τ=4.5±0.6 min, P<0.01 by F-distribution). In contrast, the A₁-selective agonist N⁶-cyclopentyladenosine (CPA) had little effect at 100 nM, and none at all at 3 μM (n=4). In FIG. 4B, at 100 nM, the A₂-selective agonist CGS-21680 exerted no effect, but the A₃-selective agonist IB-MECA again produced shrinkage (n=4, P<0.01 by F-distribution). In FIG. 4C, at high concentration (3 μM), the A₂-selective agonist CGS-21680 also triggered isosmotic shrinkage. However, preincubation of the cells with the selective A₃ receptor antagonist MRS-1191 (100 nM) abolished this effect (n=4, P<0.01, F-distribution). Solid trajectories are least-square fits with monoexponentials, whereas data sets displaying no significant shrinkage are connected by dotted lines.

FIG. 5 shows the effect of IB-MECA on short-circuit current (I_{SC}) across intact rabbit ciliary epithelium. As an initial step in data analysis, 20-min period of baseline current just before addition of any agent was fit by linear least-squares analysis. The line generated by that analysis was extrapolated to a point 45 min beyond introduction of that agent. Each current response was subtracted from its respective extrapolated baseline to yield a common initial baseline approximating constant zero current. All recordings were placed in register relative to time of agent introduction (time 0). Records of control (solvent), IB-MECA with solvent, and B-MECA corrected for solvent were separately averaged. IB-MECA was always added in the presence of 5 nM Ba²⁺ to isolate contribution of Cl⁻ to the response.

FIGS. 6A and 6B depict chemical structures. FIG. 6A depicts the structures of the physiologic agonist adenosine, the full agonist CL-IB-MECA, and nucleoside derivatives MRS-3771 and MRS-3642. FIG. 6B depicts the structure of MRS-3820, (2-(2-chloro-6-(3-iodobenzylamino)-9H-purin-9-yl)tetrahydrothiophene-3,4-diol.

### DETAILED DESCRIPTION OF CERTAIN EMBODIMENTS

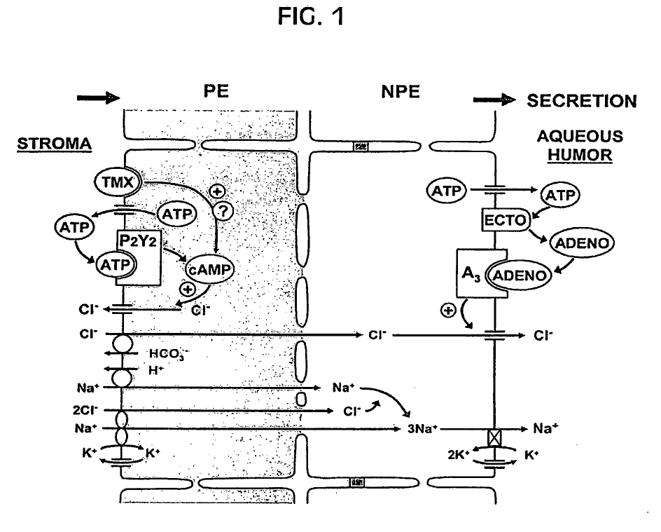

The proposed mechanism of action of A₃ receptor agonists, such as adenosine (Ado), in influencing aqueous humor secretion is shown in FIG. 1. ATP is released from PE and/or NPE cells. ATP is then converted to adenosine by ecto-enzymes (ecto). The adenosine then binds to A₃ receptors on NPE cells, resulting in opening of Cl⁻ channels. This results in an increase in aqueous humor production and increased intraocular pressure. In addition, simultaneous stimulation by ATP and tamoxifen activates Cl⁻ efflux from PE cells, leading to a net decrease in aqueous humor formation. ATP acts on P₂ receptors of PE cells, promotes opening of Cl⁻ channels, and a decrease in aqueous humor production resulting in decreased intraocular pressure.

The present invention includes the observation that the A₃ subtype adenosine receptor antagonists (referred to herein as “A₃ antagonists”) inhibit shrinkage cross-species of NPE cells as determined by measurements of cell volume in isosmotic solution. This inhibition of cell shrinkage implies a net reduction of secretion of aqueous humor through the NPE cell membrane which would result in a reduction of intraocular pressure (FIG. 1). These A₃ receptors are present on human and rabbit NPE cells and underlie the activation of NPE chloride (Cl⁻) channels by adenosine. The shrinkage of PE cells implies a stimulation of a net reabsorption of aqueous humor through the PE cell membrane towards the stroma, which would result in a net reduction in aqueous humor formation and a reduction in intraocular pressure (FIG. 1). Thus, the A₃-selective adenosine receptors increase chloride channel activity of NPE cells, and blocking these receptors by A₃ antagonists, or related compounds, reduces chloride channel activity and secretion by the NPE cells into the aqueous humor. As a result, the A₃ antagonists can be used to lower intraocular pressure as a cross-species treatment for glaucoma and other ocular conditions in which it is desirable to lower intraocular pressure.

Measurements of short-circuit current across intact rabbit ciliary epithelium, of cell volume in suspended cultured human NPE cells, and of whole-cell currents from patch-clamped cultured human and fresh bovine NPE cells have indicated that adenosine-receptor occupancy stimulates Cl⁻ secretion in mammalian NPE cells (Carre et al., supra, 1997; U.S. Pat. No. 6,528,516). As evidenced by the data presented in the examples below, these effects are mediated by A₃ receptors. A₃ receptors are present in both human HCE cells (a cell line of human NPE cells) and the rabbit ciliary body.

The A₃-selective agonist IB-MECA (N⁶-(3-iodobenzyl)-adenosine-5′-N-methyluronamide) increased the short circuit current across rabbit iris-ciliary body in the presence of Ba²⁺, a change consistent with an increased efflux of Cl⁻ from NPE cells. In the presence of gramicidin to isolate the Cl⁻ conductance, IB-MECA caused human HCE cells to shrink in a dose-dependent manner; the K_{d} of 55 nM is consistent with a maximal stimulation of A₃ receptors in cardiac myocytes at 100 nM IB-MECA (Shahidullah et al., Curr. Eye Res., 16:1006-1016, 1997). The highly specific A₃ agonist Cl⁻IB-MECA also produced shrinkage of HCE cells in the presence of gramicidin. Gramicidin readily partitions into plasma membranes to form a cation-selective pore. and is widely used for studying volume regulation (Hoffmann et al., Interaction of Cell Volume and Cell Function, Lang et al., eds., Springer, Heidelberg, Germany, pp. 188-248, ACEP Series 14 (1993)). Under these conditions, release of cell Cl⁻ becomes the rate-limiting factor in both hyposmotic (Civan et al., Invest. Opthalmol. Vis. Sci., 35:2876-2886 (1994)) and isosmotic cell shrinkage (Carre et al., supra, 1997).

Moreover, the A₃ antagonists MRS-1097 and MRS-1191 prevented the shrinkage induced by IB-MECA at concentrations far below their Kᵢ for A₁ and A_{2A} receptors. The A₁ agonist CPA did not have a consistent effect upon cell volume. The A_{2A} agonist CGS-21680 had no effect at low concentrations. The effect of CGS-21680 on shrinkage was only detected at a concentration 500 fold higher than the Kᵢ values for the A₃ receptor, and this effect was blocked by the A₃ antagonist MRS-1191. The A₃ antagonists MRS-1097, MRS-1191 and MRS-1523 blocked the shrinkage produced by 10 μM adenosine. MRS-1523, MRS-3642, MRS-3771, MRS-3826, MRS-1649 and MRS 3827 were each tested by the inventors and found to lower IOP in the mouse. Also useful are MRS 1220, and nucleosides LJ-1830, LJ-1831, LJ-1833, LJ-1834, LJ-1835LJ-1836, and LJ-1837 (all synthesized by L. S. Jeong, Korea for NIH). Consequently any derivative of a dihydropyridine, pyridine, pyridinium salt or triazoloquinazoline, expressly having A₃ receptor antagonist activity, is further contemplated within the present invention. Together, these observations indicate that the adenosine-stimulated activation of Cl⁻ release by the HCE line of human NPE cells is primarily mediated by occupancy of an A₃-subtype adenosine receptor.

In one embodiment, the A₃ antagonist MRS-3820 (2-(2-chloro-6-(3-iodobenzylamino)-9H-purin-9-yl)tetrahydrothiophene-3,4-diol), synthesized by L. S. Jeong, Korea for NIH, when non-invasively tested on normal mice, using a topically-applied droplet concentration of 250 μM (micromolar) significantly reduced intraocular pressure (IOP) within 20 minutes after the initial application. An even greater reduction was seen 30 minutes post-administration (4.2±0.7 mmHg from a baseline of 16.7±1.1, p<0.001 by paired t-test). While the point of the methods of the present invention is qualitative, rather than quantitative (time or magnitude. “Significantly” in this situation refers to a measurable IOP reduction of at least <0.05 follow, which is the conventionally accepted definition of the term. A significant change is one in which IOP is reduced by at least 5% from the pretreatment condition, or at least 10%, or at least 20%, or at least 30%, or at least 50%, or at least 60%, or at least 75%, or at least 90%, and up to 99% difference. As will be clear from the data that follow, over the periods of measurement, generally 5 to 10 minutes, or 15 min, or 20 min, or 25 min, or 30 min, or most often 35 min or longer, up to 45 min or up to 1 hour, MRS 3820 significantly lowered intraocular pressure, cross species. There was a statistically-significant change in IOP, yet the probability was less than 1 in 20 (P<0.05) that such an effect could be observed by chance alone. Moreover, the IOP reduction was applied in an expressly “species independent” application, as will be described in greater detail below, and support the initiative to deliver a species-independent, potent A₃ inhibitor to shrink non-pigmented ciliary epithelial (NPE) cells by activating Cl⁻ channels.

“Cross species” and “species independent are terms used for their ordinary meaning, i.e., that the resulting data is independent of the species of the test animal selected, and the results quite literally cross differences between species. The prodrug forms of this A₃ receptor antagonist are also contemplated for administration to the eye, which were then converted to the active antagonists, which in turn reduced intraocular pressure.

In another embodiment, the A₃ antagonist 2,4-diethyl-1-methyl-3-(ethylsulfanylcarbonyl)-5-ethyloxycarbonyl-6-phenylpyridium iodide (MRS-1649) was used to reduce intraocular pressure. The synthesis of the MRS compounds is generally described in U.S. Pat. No. 6,528,516, herein incorporated by reference. The representative MRS compound, 3,5-diacyl-1,2,4-trialkyl-6-phenylpyridinium derivative displays a uniquely high water solubility (43 mM) and can be extracted readily into ether. In addition, the prodrug form of this compound, the corresponding 1-methyl-1,4-dihydropyridine, can be oxidized to form compound MRS-1649 in vitro in the presence of a tissue homogenate. Thus, it is contemplated that prodrug forms of A₃ receptor antagonists can be administered to the eye which will then be converted to the active antagonists which will reduce intraocular pressure.

In addition to the particular A₃ receptor antagonists discussed in the examples below: MRS-1097 (3-ethyl 5-benzyl-2-methyl-6-phenyl-4-styryl-1,4-(±)-dihydropyridine-3,5-dicarboxylate), MRS-1191 (3-ethyl 5-benzyl-2-methyl-6-phenyl-4-phenylethynyl-1,4-(±)-dihydropyridine-3,5-dicarboxylate) (Jiang et al., J. Med. Chem. 40:2596-2608 (1997)), MRS-1523 (Li et al., J. Med. Chem. 42:706-721 (1999)), and MRS-3820 (2-(2-chloro-6-(3-iodobenzylamino)-9H-purin-9-yl)tetrahydrothiophene-3,4-diol), the use of any A₃ receptor antagonist or analog thereof to reduce intraocular pressure is within the scope of the invention. Other A₃ receptor antagonists for use in the present invention are described by Jacobson (Trends Pharmacol. Sci. 19:184-191 (1998)) and include MRS-1334 (3-ethyl 5-(4-nitrobenzyl) 2-methyl-6-phenyl-4-phenylethynyl-1,4-(O)-dihydropyridine-3,5-dicarboxylate)), MRS1067 (3,6-dichloro-2′-(isopropoxy)-4′-methylflavone), MRS-1220 (9-chloro-2-(2-furyl)-5-phenylacetylamino-[1,2,4]-triazolo[1,5-c]quinazoline), L249313 (6-carboxymethyl-5,9-dihydro-9-methyl-2-phenyl-[1,2,4]-triazolo[5,1-a][2,7]naphthyridine) and L268605 (3-(4-methoxyphenyl)-5-amino-7-oxo-triazolo[3,2]pyrimidine), VUF8504 (4-methoxy-N-[2-(2-pyridinyl)quinazdin-4-yl]benzamide) and the like.

The data presented below demonstrate the ability of various cross-species agents to block shrinkage of NPE cells and to promote shrinkage of PE cells. The net effect of these agents would be to reduce intraocular pressure in vivo. Also contemplated within the subject matter of the present invention is the use of four chemical classes of A₃ receptor antagonists for reduction of intraocular pressure: dihydropyridines (e.g., MRS 1097, MRS 1191), pyridines, pyridinium salts (e.g., Compound 23 (Xie et al., J. Med. Chem. 42:4232-4238 (1999))) and triazoloquinazolines (e.g., MRS1220 (Pugliese et al., Br. J. Pharmacol. 147:524-532 (2006))), and derivatives thereof having A₃AR antagonist activity (e.g., triazoquinazoline derivative: MRS 1220; and nucleosides LJ-1830, LJ-1831, LJ-1833, LJ-1834, LJ-1835, LJ-1836, and LJ-1837). These classes of compounds are also described in PCT/W097/27177; and U.S. Pat. No. 6,528,516.

The determination of whether a compound can act as an A₃ receptor antagonist can be determined using standard pharmacological binding assays. However, when tests were initiated to demonstrate that the IOP-reducing effect of an A₃AR antagonist is independent of the species being treated, methods were needed to permit reliable determination of changes in IOP on the small mouse eye. The effects of A₃AR antagonists on mouse IOP were measured by the invasive servo-null technique developed by the inventors for the small mouse eye, and which requires impalement of the cornea with a fine, hollow glass needle, whose tip diameter is about 5 micrometers. Using the invasive servo-null technique (Avila et al., supra, 2001), the relatively large reduction in IOP (of about 5 mm Hg) in normal mice has suggested that as demonstrated above, A₃ antagonists are indicated as in vivo therapeutic compounds for treating patients with glaucoma. However, two additional observations were noted. First, topical application of an A₃AR-antagonist provided only a very slight decrease in IOP in monkeys (Okamura et al., Bioorg. Med. Chem. Lett. 14(14):3775-3779 (2004)), but those measurements were conducted non-invasively, without puncturing the cornea. Second, the response of mouse IOP to topical application of A₃AR-antagonists was much more rapid in mice measured with the invasive servo-null technique, as compared with other mammals (Avila et al., supra, 2001, 2002; Pang et al., Exp. Eye Res. 80(2):207-214 (2005)). This second finding is addressed in greater detail in Example 1, below.

Lowering of intraocular pressure with a combination of an antiestrogen and ATP, or any compound capable of promoting ATP release from NPE cells, is also contemplated, alone or in combination, including combinations with A₃ antagonists. The use of a calmodulin antagonist for lowering intraocular pressure is also within the scope of the invention including, but not limited to calmidazolium chloride, calmodulin binding domain, chlorpromazine HCl, melittin, phenoxybenzamine HCl, trifluoperazine dimaleate, W-5, W-7, W-12 and W-13. These compounds are available from Calbiochem, San Diego, Calif. The use of analogs of the above-identified compounds for the reduction of intraocular pressure is also within the scope of the present invention.

These agents can be used to treat ocular disorders resulting associated with, or caused by, an increase in intraocular pressure, such as glaucoma. The agents can be processed in accordance with conventional methods to produce medicinal agents for administration to mammals or other animals subject to increased IOP, preferably to humans. The intended patients or subjects (collectively referred to herein as “individuals”) of the present invention include any animal or human subject to, or predisposed to, increased IOP of the eye of the type resulting in the disease state recognized as glaucoma.

The agents can be employed in admixture with conventional excipients, i.e. pharmaceutically acceptable organic or inorganic carrier substances suitable for parenteral, enteral (e.g., oral) or topical application which do not deleteriously react with the agents. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrollidone, etc. The pharmaceutical preparations can be sterilized and, if desired, mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, particularly suitable are injectable, sterile solutions, preferably oily or aqueous solutions, as well as suspensions emulsions, or implants, including suppositories. Ampules are convenient unit dosages. For enteral application, particularly suitable are tablets, liquids, drops, suppositories or capsules. A syrup, elixir or the like can be used when a sweetened vehicle is employed. Sustained or directed release compositions can be formulated, e.g. liposomes or those wherein the active compound is protected with differentially degradable coatings, e.g. by micro-encapsulation, multiple coatings, etc. It is also possible to lyophilize the agents for use in the preparation of products for injection.

For topical application, there are employed non-sprayable forms, viscous to semi-solid or solid forms comprising a carrier compatible with topical application and having a dynamic viscosity preferably greater than water. Suitable formulations include, but are not limited to, solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, ocular permeability, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., freon.

In a preferred embodiment, the agent is formulated into a pharmaceutical formulation appropriate for administration to the eye, including eye drops, gels and ointments. See also the recently discovered barrier effects of the corneal membrane, resulting in blocked or inhibited passage of the topically applied drug to the target area as recently reported by Wang et al., Experim. Eye Research 85:105-112 (2007), which may have to be considered by medical personnel in the delivery of the IOP-relieving drugs to the eye of an animal or human patient.

For systemic administration, the dosage of the agents according to this invention generally is between about 0.1 μg/kg and 10 mg/kg, preferably between about 10 μg/kg and 1 mg/kg. For topical administration, dosages of between about 0.000001% and 10% of the active ingredient are contemplated, preferably between about 0.1% and 4%. It will be appreciated that the actual preferred amounts of agent will vary according to the specific agent being used, the severity of the disorder, the particular compositions being formulated, the mode of application and the species being treated. Dosages for a given host can be determined using conventional considerations, e.g., by customary comparison of the differential activities of the subject compounds and of a known agent, e.g., by means of an appropriate, conventional pharmacologic protocol. The agents are administered from less than once per day (e.g., every other day) to four times per day.

The invention is further defined by reference to the following specific, but non-limiting examples. Reference is made to standard textbooks of molecular biology that contain definitions and methods and means for carrying out basic techniques, encompassed by the present invention. It will be apparent to one skilled in the art that many modifications, both to materials and methods, may be practiced without departing from the purpose or narrowing the scope of this invention.

### EXAMPLES

In the examples that follow, two issues are address in Examples 1 through 3, respectively. First, is a determination of whether the cornea presents a substantial barrier to the therapeutic delivery of IOP-reducing drugs to the interior of the eye, when such drugs are topically applied as drops to the tear film. Second, is a determination of whether there is a substantial species variation in the response of A₃ subtype adenosine receptors to the administered antagonists, or whether the response is independent, since without such confirmation, a favorable response in a laboratory rodent would not necessarily ensure a similarly favorable effect in a human. In the examples that follow certain materials and methods are used, often in a manner that corresponds to the materials and methods associated with previously reported experiments, such as those reported in U.S. Pat. No. 6,528,516.

In general, values are presented as the means±1 SE. The number of experiments is indicated by the symbol N. The null hypothesis, that the experimental and baseline measurements shared the same mean and distribution, was tested with Student's t-test and by the upper significance limits of the F-distribution, as indicated. The t-test was applied to compare the significance between single means or single fit parameters. The F-distribution was applied to test whether the time course of volume measurements in different suspensions could reflect a single population of data points.

Materials: Gramicidin, adenosine, 2-chloroadenosine, tamoxifen, ATP, 17α- and β-estradiol, DiC₈, carbachol, atropine, histamine, and trifluoperazine were obtained from the Sigma Chemical Co. (St. Louis, Mo.). CPA (N⁶-cyclopentyl-adenosine), CGS-21680, IB-MECA, Cl-IB-MECA and MRS-1191 (3-ethyl 5-benzyl 2-methyl-6-phenyl-4-phenylethynyl-1,4-(±)-dihydropyridine-3,5-dicarboxylate) were obtained from Research Biochemicals International (Natick, Mass.). Fura-2 AM was bought from Molecular Probes (Eugene, Oreg.). MRS-1097, MRS-1523 and MRS-3820 were provided by Drs. Kenneth A. Jacobson (National Institutes of Health) and Bruce L. Liang (University of Pennsylvania). The compound Cl-IB-MECA (MH-C-7-08; Lot No. CMVIII-12) was provided by Research Biochemicals International as part of the Chemical Synthesis Program of the National Institute of Mental Health, Contract N01MH30003. DIDS [4,4′-diisothiocyano-2,2′-disulfonic acid] and fura-2 AM were obtained from Molecular Probes, Inc. (Eugene, Oreg.). NPPB [5-nitro-2-(3-phenylpropylamino)benzoate] and staurosporine were obtained from Biomol Research Laboratories, Inc. (Plymouth Meeting, Pa.).

Cell Culture The HCE (human ciliary epithelial) cell line (Carre et al., supra, 1997) is an immortalized NPE cell line obtained from primary cultures of adult human epithelium. Cells were grown in Dulbecco's modified Eagle's medium (DMEM, #11965-027, Gibco BRL, Grand Island, N.Y.) with 10% fetal bovine serum (FBS, A-1115-L, HyClone Laboratories, Inc., Logan, Utah) and 50 μg/ml gentamycin (#15750-011, Gibco BRL), at 37° C. in 5% CO₂ (Wax et al., Exp. Eye Res. 57:3057-3063 (1993); Yantorno et al., Exp. Eye Res. 49:423-437 (1989)). The growth medium had an osmolality of 328 mOsm. Cells were passaged every 6-7 days and were studied 8-13 days after passage, after reaching confluence. An immortalized PE-cell line from a primary culture of bovine pigmented ciliary epithelium were also grown under matching conditions.

Measurement of Cell Volume in Isosmotic Solution: The volume of PE and NPE cells was measured, since the movement of fluid underlies a change in PE and NPE cell volume, respectively. This is also thought to be the same as the movement of fluid which underlines the secretion of aqueous humor (FIG. 1).

After harvesting the cells from a single T-75 flask by trypsinization (Yantorno et al., supra), 0.5-ml aliquot of the HCE cell suspension, or of the bovine cell suspension, in DMEM (or in Cl⁻-free medium, where appropriate), was added to 20 ml of each test solution, which contained (in mM): 110.0 NaCl, 15.0 HEPES [4-(2-hydroxyethyl)-1-piperazineethanesulfonic acid], 2.5 CaCl₂, 1.2 MgCl₂, 4.7 KCl, 1.2 KH₂PO₄, 30.0 NaHCO₃, and 10.0 glucose, at a pH of 7.4 and osmolality of 298-305 mOsm. Parallel aliquots of cells were studied on the same day. One aliquot usually served as a control, and the others were exposed to different experimental conditions at the time of suspension. The same amount of solvent vehicle (dimethylformamide, DMSO or ethanol) was always added to the control and experimental aliquots. The sequence of studying the suspensions was varied to preclude systematic time-dependent artifacts (Civan et al., Exp. Eye Res. 54:181-191 (1992); Civan et al., 1994).

Cell volumes of isosmotic suspensions were measured with a Coulter Counter (model ZBI-Channelyzer II), using a 100 μm aperture (Civan et al., supra, 1994). As previously described (Wax et al., supra, 1993), the cell volume (v_{C}) of the suspension was taken as the peak of the distribution function. Cell shrinkage was fit as a function of time (t) to a monoexpenential function:

v_{C}=v₂₈+(v₀v_{∞})−[e^{-(t-t}^{₀}^{)/τ}] {1)

where v_{∞} is the steady-state cell volume, v₀ is the cell volume at the first point (t₀) of the time course to be fit, and τ is the time constant of the shrinkage. For purposes of data reduction, the data were normalized to the first time point, taken to be 100% isotonic volume. Fits were obtained by nonlinear least-squares regression analysis, permitting both v_{∞} and τ to be variables.

In previous studies demonstrating that adenosine causes isotonic cell shrinkage by activating Cl⁻ channels in NPE cells (Carre et al., supra, 1997), the levels of adenosine used were sufficiently high to activate A₁, A_{2A}, A_{2B} or A₃ adenosine receptor subtypes In order to differentiate among these receptors, the experiments were repeated using a series of agonists and antagonists selective for these receptors. In the presence of gramicidin, the A₃ agonist IB-MECA caused the cells to shrink in a concentration-dependent manner. The apparent K_{d} for the IB-MECA-induced shrinkage was 55±10 nM. IB-MECA is a highly selective agonist for the A₃ receptor, wherein the reported Kᵢ for the A₃ receptor is 50 times lower than it is for the A₁ or A_{2A} receptor (Gallo-Rodrigez et al, J. Med. Chem. 37:636-646 (1994; Jacobson et al., supra, 1995; Jacobson et al., FEBS Lett. 336:57-60 (1993)).

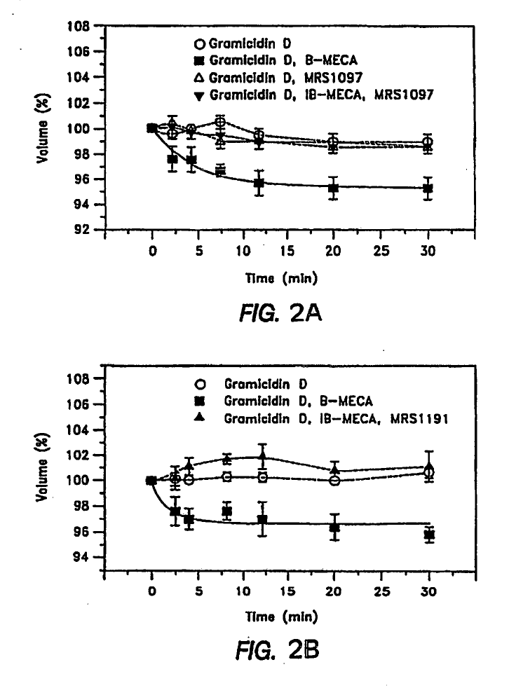

It was also determined whether A₃-selective antagonists could prevent the putative A₃-mediated shrinkage produced by IB-MECA. Parallel aliquots of suspensions were preincubated with MRS-1097, a selective A₃-selective antagonist with Kᵢ values for the binding (in nM) to human A₁/A_{2A}/A₃ receptors of 5,930/4,770/108 (Jacobson et al., Neuropharmacol. 36:1157-1165 (1997)). Preincubation for 2 min with 300 nM MRS-1097 blocked the isomotic shrinkage characteristically triggered by 100 nM IB-MECA (FIG. 2A). A second highly selective A₃ antagonist, MRS-1191, (Jiang et al., J Med. Chem. 39:4667-4675, 1996), with Kᵢ values for the binding (in nM) to human A₁/A_{2A}/A₃ receptors of 40,100/>100,000/31.4 was also used. Preincubation for 2 min with 100 nM MRS-1097 also prevented the subsequent response to 100 nM IB-MECA (FIG. 2B).

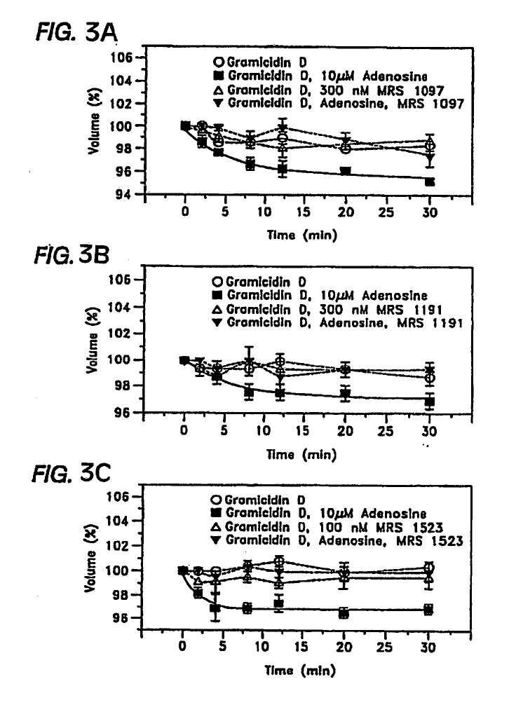

The physiologic agonist reaching the adenosine receptors is likely to be the nucleoside adenosine itself, arising from release of ATP by the ciliary epithelial cells and ecto-enzyme activity (Mitchell et al., supra, 1998). Adenosine triggers isosmotic shrinkage of cultured human NPE cells with an EC₅₀ of 3-10 μM (Civan et al., supra, 1997). In this concentration range, adenosine also acts as a nonselective agonist of all four subtypes of the adenosine receptor ((Fredholm et al., Pharmacol. Rev. 46:143-156 (1994); Fredholm et al., Trends Pharmacol. Sci. 18:79-82 (1997)). As illustrated in FIG. 3, a 2 min preincubation with either 100 nM of the A₃-selective antagonist MRS-1191 (FIG. 3B), or 300 nM of the A₃-selective antagonist MRS-1097 (FIG. 3A), blocked the shrinkage characteristically produced by 10 μM adenosine. MRS-1523, an A₃ antagonist with Kᵢ values for the binding (in nM) to human A₁/A_{2A}/A₃ receptors of 15,600/2,050/19 (Li et al., J. Med. Chem. 41:3186-3201, 1998) also eliminated the actions of adenosine.

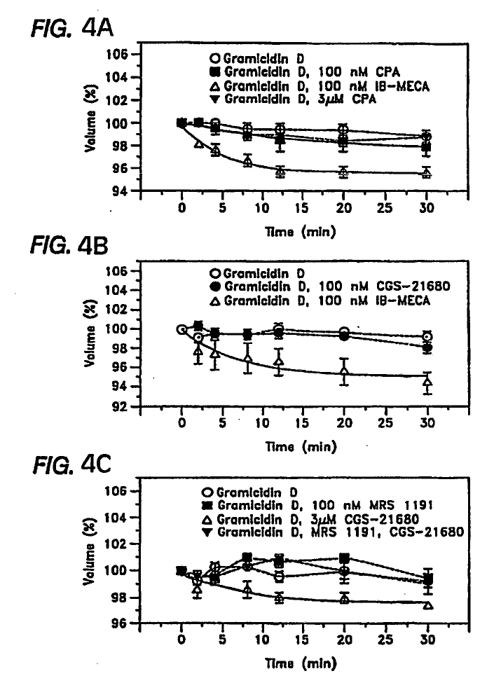

Thus, the ability of specific A₃ antagonists to inhibit the response to the nonspecific adenosine suggests that the contribution of the other receptors to Cl⁻ channel activation was minimal. To test this further, the effect of A₁ and A_{2A} agonists were tested. CPA is an A₁-selective agonist with a Kᵢ for the A₁-receptor of 0.6 nM. However, CPA produced no significant shrinkage at 30 nM and 1 μM (data not shown, N=3) and 3 μM (FIG. 4A). A small slow effect of uncertain significance was detected at the intermediate concentration of 100 nM (FIG. 4A). Some cross-reactivity with A₃ receptors might be expected, given the Kᵢ of CPA for the A₃-subtype of 43 nM (Klotz et al., supra). CGS-21680 is a widely used A_{2A} agonist with an IC₅₀ value of 22 nM for the A_{2A}-receptor (Hutchison et al., J. Pharmacol. Exp. Ther. 251:47-55, 1989, Jarvis et al., J. Pharmacol. Exp. Ther. 253:888-893, 1989). CGS-21680 had no detectable effect at 100-nM concentration (FIG. 4B), but did trigger isosmotic shrinkage at a 30-fold higher concentration (3 μM) (FIG. 4C).

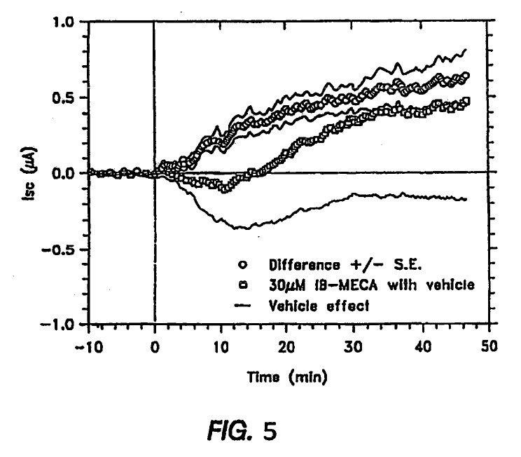

However, the Kᵢ for the CGS-21680 at the A₃ receptor is 67 nM (Klotz et al., supra), and thus, CGS-21680 could have been acting though either A_{2A} receptors or A₃ receptors at the higher concentration. To distinguish between these possibilities, parallel aliquots of suspensions were preincubated with the antagonist 100 nM MRS-1191. MRS-1191 prevented the shrinkage produced by the high concentration of CGS-21680 (FIG. 5C, P<0.01, F-test), indicating that the shrinkage observed was mediated by cross-reactivity with A₃ receptors. As there are presently no high-affinity A_{2B} agonists (Klotz et al., supra), the contribution of A_{2B} receptor stimulation was not pursued, although the ability of A₃ antagonists to inhibit the response to 10 μM adenosine (FIG. 4) argues against a role for the A_{2B} receptor. For example, MRS1191 at 10 μM did not displace radioligand binding to recombinant human A_{2B} receptors, thus it is a truly selective A₃ antagonist.

Transepithelial Measurements: In animal experiments, preferably rabbits, after anesthetization and sacrifice (Carre et al., J. Membr. Biol. 146:293-305 (1995), the iris-ciliary body (1-CB) was enucleated and isolated as described by Carre et al., 1995. In one experiment, the pupil and central iris were occluded with a Lucite disc, and the iris-ciliary body was mounted between the two halves of a Lucite chamber. The annulus of exposed tissue provided a projected surface area of 0.93 cm². Preparations were continuously bubbled with 95%O₂-5% CO₂ for maintenance of pH 7.4 in a Ringer's solution comprising (in mM): 110.0 NaCl, 10.0 HEPES (acid), 5.0 HEPES (Na⁺), 30.0 NaHCO₃, 2.5 CaCl₂, 1.2 MgCl₂, 5.9 KCl, and 10.0 glucose, at an osmolality of 305 mOsm. BaCl₂ (5 mM) was added to the solution to block K⁺ currents. The transepithelial potential was fixed at 0 mV, corrected for solution series resistance, and the short-circuit current was monitored on a chart recorder. Data were digitally acquired at 10 Hz via a DigiData 1200A converter and AxoScope 1.1 software (Axon Instruments, Foster City, Calif.). Automatic averaging was performed with a reduction factor of 100 to achieve a final sampling rate of 6/min.

Correcting the Possible Solvent Effect: Adenosine in high concentration (100 μM) has been found to increase the short-circuit current across the rabbit ciliary body (Carre et al., supra, 1997). Therefore, a high concentration (30 μM) of the A₃ agonist IB-MECA was tested to determine if it also affected short-circuit current. At this concentration, the vehicle (dimethylformamide) itself exerts significant effects (FIG. 5, lowest trajectory). The solvent effect was corrected as follows: solvent alone was initially introduced (to 0.1%), followed by the same volume of solvent (to 0.2%) containing agonist, and ending with addition of a third identical volume of solvent alone (to a final concentration of 0.3%). The reduction in short-circuit current following the first addition of solvent was always greater than the third. In each of four experiments, the time courses of the first and third additions were averaged to estimate the effect of raising the solvent concentration without agonist from 0.1% to 0.2% during the experimental period. FIG. 5 presents the mean trajectory for the averaged solvent effect, the uncorrected mean time course following exposure to IB-MECA, and the mean trajectory±1 SEM for the solvent-corrected response. The experiments were performed in the presence of 5 mM Ba²⁺ to minimize the contribution of K⁺ currents. IB-MECA produced a significant increase in the short-circuit current; an increase in short-circuit current in the presence of Ba²⁺ suggesting that the effect is mediated by activating a Cl⁻ conductance on the basolateral membrane of the NPE cells. The sustained nature of the stimulation is consistent with the time course of the cell shrinkage in response to A₃ stimulation.

### Example 1

### Corneal Barrier to Delivery of Topical Drugs to Targets within the Eye

The effects of A₃AR antagonists on mouse IOP have traditionally been measured by the invasive servo-null technique developed by the inventors for the small mouse eye, and which requires impalement of the cornea with a fine, hollow glass needle, whose tip diameter is about 5 micrometers (Avila et al., supra, 2001, 2002) However, in order to demonstrate the in vivo IOP-reducing effect of the A₃AR-antagonist compounds, the testing techniques were refined. A pneumotonometer was adapted for measuring mouse IOP non-invasively (Avila et al., Invest. Opthalmol. Vis. Sci. 46:3274-3280, 2005)). Using this technique, it was found that it took 30 minutes for the A₃AR-antagonist (MRS-1191) to begin lowering mouse IOP significantly after topical application to the eye, whereas the same drug begins to lower IOP within about a minute when IOP is measured with the invasive servo-null technique. Likewise, the increase in IOP triggered by the non-selective AR-agonist adenosine was delayed by about 10 minutes when measured non-invasively. These results provided a strong indication that the rapid response of mouse IOP to topically applied A₃AR-antagonists was actually a result of drug entry through damaged tissue around the micropipette tip, although the same effects were observed after much slower delivery of the drugs by diffusion across the very thin cornea of the mouse (about 170 micrometers in depth).

To verify this effect, an entirely different parameter was monitored in the test animals. The barrier properties of the mouse eye by monitoring (1) pupil size following topical application of carbachol (a miotic agent) and (2) intraocular pressure (IOP) responses to purinergic drugs measured by both the invasive servo-null micropipette system (SNMS) and non-invasive pneumotonometry.

The test animals were black Swiss outbred mice of mixed sex, 7-9 weeks old and 25-30 g in weight, obtained from Taconic Inc. (Germantown, N.Y.), and maintained under 12:12-h light/dark illumination cycle and allowed unrestricted access to food and water. Mice were anesthetized with intraperitoneal ketamine (250 mg k⁻¹) supplemented by topical proparacaine HCl 0.5% (Allergan, Bausch & Lomb) for the IOP measurements. IOP was measured invasively (SNMS) and non-invasively by pneumotonometry in separate animals.

To measure pupil diameter, the pupil and an adjacent ruler having 1-mm graticules were imaged with a digital camera. Care was taken to avoid applying mechanical stress, and consequently to avoid displacing the micropipette tip from its position in the anterior chamber. Lengths were measured by IMAGE J (National Institutes of Health) and the pupil diameters were calibrated to the ruler.

By the SNMS approach (Avila et al., supra, 2001), the exploring micropipette, of 5-10 mm outer diameter, was filled with a highly conducting solution and advanced across the cornea into the anterior chamber. A refined technique by Wang et al., Invest. Opthalmol. Vis. Sci. (serial online) (Aug. 31, 2006) available at www.iovs.org/egi/letters/46/9/3274#422, enhanced stability and reduce the background noise of the records, permitting fabrication of micropipettes whose resistance is 0.1-0.3 MΩ, rather than the 0.25-0.4 MΩ used initially. The filling solution was reduced from 3M KCl to 2M NaCl, rather than 3 M KCl. The resistance of the filled micropipette was balanced in a bridge circuit, and the tip was then advanced across the cornea. Upon entering the anterior chamber of the eye, the IOP forces the much lower-conducting aqueous humor into the micropipette tip, displacing the original filling solution. The micropipette resistance was thereby increased, unbalancing the bridge circuit and triggering a bellows to provide a counter-pressure, restoring the position of the filling solution and returning the resistance to its initial value. Thus, the value of the counter-pressure equals the IOP. As in the past (Avila et al., supra, 2001), the stability of the records permitted continuous measurements for tens of minutes during the course of drug applications.

By comparison, IOP was measured non-invasively by pneumotonometry (OBT) (Avila et al., supra, 2005). As previously described, the commercially available tip of the ocular blood tomography (OBT) pneumotonometer (Blood Flow Analyzer [BFA] probe tip; Paradigm Medical Industries Inc.) was fit to a custom-built mount. Air flow from a constant pressure source was passed through the mount to reach a diaphragm forming the end of the BFA tip. The flow of air displaces the diaphragm outward, permitting escape of the air through holes in the wall of the probe tip into the atmosphere. Pressure was monitored with a transducer connected through a T-connection to the base of the BFA tip. The probe assembly was advanced to the cornea with a three-axis micromanipulator. The probe tip was advanced sufficiently to make contact with the tear film, as was indicated by a shift in the baseline output reading. In a refined method, the tip was withdrawn until the micropipette tip was visually displaced from the tear film. The output was then adjusted to zero before advancing the tip again. Contact with the cornea depresses the diaphragm of the BFA tip, occluding access of the air flow to the escape holes and raising the pressure at the base of the tip. The increase in pressure with advance of the probe characteristically displays a relative plateau or inflection region, which is taken to be the endpoint for the IOP.

This approach was simplified to expedite identification of the endpoint, and the probe was subsequently advanced in about 10 standardized steps of approximately 50 mm at intervals of about 10 seconds to identify the inflection region (Wang et al., supra, 2006). In either case, the endpoint was considered technically acceptable if the pressure recording also displays oscillations of pressure clearly in synchrony with the simultaneously measured cardiac pulse. The pneumotonometric estimates of IOP were previously found to agree with manometric measurements in cannulated preparations and with estimates obtained by the servo-null technique (Avila et al., supra, 2005). Having established the baseline value of IOP, the position along the axis of advance of the micromanipulator was noted, and the probe was retracted from contact with the cornea. Subsequent measurements of IOP at later time points were obtained by advancing the probe tip to the same position, with the placement of the mouse maintained stereotactically. The measurements were conducted at 10-min intervals to avoid potential artifacts associated with prolonged pressure on the cornea. Following this protocol, control measurements displayed great stability over the more than 30 min of the period of measurement).

The cardiac rate was monitored with a pressure transducer wrapped around the tail (MLT1010, Adinstruments, USA). Both IOP and cardiac pulse signals were band-pass filtered (1-100 Hz), amplified using a signal conditioner (CyberAmp 380, Axon Instruments Inc., USA) and then digitized at 1 kHz using an analog-to-digital converter (MiniDigi 1A two-channel acquisition system, Axon Instruments Inc., USA) in the gap-free mode. The resulting digital files were analyzed off-line using Clampfit 9 (Axon Instruments).

Ketamine HCl was purchased from Phoenix Pharmaceutical Inc. (St. Joseph, Mo.). Other drugs were obtained from Sigma Chemical (St. Louis, Mo.). Drugs were applied topically with an Eppendorf pipette. MRS-1191 and Cl-IB-MECA were initially dissolved in DMSO and then added to a saline solution containing benzalkonium chloride to enhance corneal permeability. The final droplet solution contained the drugs at the stated concentrations together with <2% DMSO and 0.0005% benzalkonium chloride at an osmolality of 295-300 mOsm. DMSO was omitted, altogether, from droplets containing the hydrophilic compounds adenosine, carbachol and carboxyfluorescein.

Effects on invasively-measured IOP were measured 10 min after topical application because after this time, the continued presence of the micropipette can be associated with a downward drift of the IOP, even under control conditions. In contrast, smaller droplets (5 ml instead of 10 ml) were applied to the eye more frequently (three times instead of once) with the non-invasive technique, in order to forestall drying of the eye associated with the airflow from the pneumotonometer. With the non-invasive protocol, the IOP was stable for 30 min under control conditions.

Pupillary diameter was measured before and 10 min after topical addition of 10-ml droplets containing 40 mM (0.073 mg) of the miotic carbachol to both eyes of the mouse. One eye was not punctured; the other eye was impaled with the SNMS micropipette. Either the right or left eye of each mouse was chosen randomly for impalement. At so low a topical concentration and in the absence of a micropipette, exposure to carbachol for 10 min had no significant effect on pupillary size. The baseline diameter was 1.80±0.11, and was insignificantly changed by carbachol application, increasing by 0.12±0.17 mm (n=6, P>0.4). In contrast, with impalement of the cornea of the other eye with a micropipette, topical carbachol reduced the pupil diameter by 38%, contracting by 0.76±0.09 mm from a baseline of 2.00±0.15 mm (n=6, P<0.0005). At a ten-fold lower droplet concentration and dose (4 mM and 7.3 ng), carbachol had no effect on pupil size, with or without corneal impalement (data not shown; n=2). The results indicate that even the corneal perforations produced by fine-tipped micropipettes used for SNMS tonometry can facilitate drug delivery from the tear film to intraocular target sites.

Whether drug penetration into the eye was influenced by corneal impalement by a micropipette was further tested by topically applying a 10-ml droplet containing 0.003% carboxyfluorescein (0.3 mg) to both eyes of two mice. Carboxyfluorescein is a highly polar molecule that crosses the barrier layers of the eye poorly. In each mouse, an exploring micropipette was first advanced into the aqueous humor of one eye while the companion eye was not impaled. After 5 min, the dye was washed out with isotonic saline. Green fluorescence was observed in the anterior chamber of the impaled mouse eyes but not in the control eyes, again suggesting that corneal perforations with micropipettes can facilitate transfer of drugs and chemicals from the tear into the aqueous humor.

Topical application of the non-selective AR agonist adenosine, 10 mM in a droplet volume of 10 ml (26.7 mg), promptly increased mouse IOP. The peak response was reached within several minutes and the mean±S.E.M. increase over baseline after 10 min was 24.0±4.7 mmHg (n=14, P<0.001). Similarly, the selective A3 agonist Cl-IB-MECA (200 nM, 1.09 ng) elevated IOP by 10.0±2.9 mmHg (n=9, P<0.01). The established selective dihydropyridine A3 antagonist MRS-1191 exerted an opposite effect. At a droplet concentration of 2.5 mM (11.94 mg), MRS-1191 reduced IOP by 6.1±1.1 mmHg, again over a period of several minutes following application (n=6, P<0.001).

When the noninvasive pneumotonometric measurements of IOP were tested in response to adenosine, Cl-IBMECA and MRS-1191 (droplets of 5111 each) in the absence of corneal impalement, the detectable increase was slower and smaller (P<0.02) than that measured by the invasive servo-null technique. The difference between the SNMS and pneumotonometric measurements was even more striking with the selective A3 agonist Cl-IB-MECA, which produced no significant increase in IOP. The pneumotonometrically measured decrease in IOP triggered by the established A3 antagonist MRS-1191 was closer to that detected by the SNMS. However, the maximum decrease observed pneumotonometrically was observed at the end of the experiments, 30 min after the initial application of MRS-1191. In contrast, the maximum effect of the MRS-1191 detected by the SNMS was much earlier, at 9.6-1.1 min.

The salient findings of this experiment were that: (1) topical application of 40 mM (0.073 mg) carbachol produced rapid miosis following corneal impalement with a micropipette, but not following topical application without corneal impalement; and (2) topical administration of the agonists adenosine and Cl-IB-MECA and antagonist MRS-1191 trigger smaller, slower IOP effects measured non-invasively by pneumotonometry than measured invasively by SNMS tonometry. While multiple factors could be involved including the thinness of the murine cornea, it was eventually determined by the experiments presented herein, that the IOP-measuring technology, itself, plays a major role in the delivery of the topically applied therapeutic compound to the anterior chamber. Even though the SNMS approach involves a fine exploring micropipette, whose diameter is 5-10 mm, some 5-10-times smaller than that of the microneedle used for the conventional manometric technique, the impalement of the corneal significantly changes the delivery of the drug to its target.

The fineness of the tip minimizes leak, but parallel IOP measurements by invasive and non-invasive techniques demonstrate that the ocular coats of the mouse eye, despite their thin structure, present a substantial barrier to drug penetration. The results obtained with carbachol and purinergic drugs document that drug delivery is enhanced by micropipette impalement of the cornea. All of the presently studied purinergic drugs exerted rapid, large effects on mouse IOP if applied topically during corneal impalements, but display highly variable rates of action when applied to the untreated eye. The ocular permeability of these purinergic drugs was not a simple function of relative hydrophobicity. By making complementary measurements of mouse IOP by SNMS tonometry substantially facilitates delivery of the drug through the corneal barrier or ocular coats, and enhances drug efficacy, even if topical drug penetration is too slow to manifest convincing physiologic effects in intact eyes.

### Example 2

### Species Independent Effect of A₃AR-Antagonists In Vivo in an Animal Model

The A₃AR-antagonists used in previous studies both blocked adenosine-triggered shrinkage of cultured human NPE cells and lowered IOP of mice. The NPE cells were from clone-4, derived from a primary culture of human non-pigmented ciliary epithelium (see, Martin-Vasallo et al., J Cell Physiol. 141:243-252 (1989)). However, the heterocyclic derivatives, such as the dihydropyridine MRS-1191 and the pyridine MRS-1523, used in those studies, displayed widely varying affinities for the A₃AR of different species. This variability is illustrated by the very high binding affinities of the antagonists to rat, relative to human A₃ receptors, ranging from 10 to >30,000 (Yang et al., supra, 2005). The non-generality of antagonist selectivity, therefore, had limited the ability to evaluate the potential clinical relevance of the known A₃ antagonists in animal models, in which the A₃ selectivity of the compounds could be very different from the human.

To address this affinity issue, A₃ antagonists were constructed by modifying the A₃ agonists, whose high affinity of IB-MECA at A₃ receptors extends across species. See previously verification that A₃ antagonists, e.g., MRS-1292 (Gao et al., Biochem. Pharmacol. 65:1675-1684 (2003)), is effective both in blocking adenosine-triggered shrinkage of cultured human NPE cells and in lowering IOP of mice (Yang et al., supra, 2005). MRS-1292 was tested on the mouse for two reasons. Transgenic mice provide a convenient opportunity for studying the molecular physiology and pharmacology in the living animal (e.g., Avila et al., supra, 2003). Second, measurement of IOP permitted us to assess the effects of MRS-1292 on the target parameter. However, recognized adenosine-stimulation protocols were not used in the mouse because it not only stimulates A3 receptors, but also activates other ARs that have independent effects on IOP. Therefore, to directly assess the effect of MRS-1292 in vivo in the mouse, IOP was monitored before and after drug application. IOP was monitored with the Servo-Null-Micropipette System (SNMS). The test protocols are described in detail by Yang et al. supra, 2005, herein incorporated by reference.

MRS 1292 is a nucleoside derivative structurally related to the agonist IB-MECA, whose high affinity of IB-MECA at A3 receptors extends across species. MRS 1292 is also an A₃-receptor-selective in both the human and the rat. Notably, the ratio of rat-to-human affinities for A₃ receptors is similar for MRS-1292 and selective A₃ agonists. When MRS-1292 was tested by Yang et al, supra. 2005, it was found to operate as an A₃ adenosine-receptor antagonist in mimicking effects of non-purine A₃ antagonists on cultured human NPE cells and altered mouse IOP. Specifically, cultured human NPE cells, pretreated with the antagonists for 2 min before initiating the measurements, were suspended in control solution containing gramicidin displayed slight shrinkage over the 60 min study (Δv_{∞}=1.2±0.1%). The symbol Δv_{∞} symbolizes the steady-state shrinkage. Adenosine (10 μM) increased the degree of shrinkage several-fold. MRS-1292 significantly reduced the magnitude (Δv_{∞}=1.9±0.2%, p<0.001 by Student's t test) and slowed the rate of the adenosine triggered shrinkage. In the presence of MRS-1292, the time constant (τ) of the shrinkage was prolonged from 3.8±0.6 to 11.7±2.6 min (p<0.02). In the presence of the 1,4-dihydropyridine A3 antagonist MRS-191, adenosine-treated cells displayed no exponential shrinkage. As noted previously, MRS-1191 has been previously used successfully to antagonize human, rat, and mouse ARs.

Topical addition of droplets containing 25 μM of the putative antagonist MRS-1292 produced a maximum reduction in IOP by 8 to 19 min (mean 15±1 min) of 4.4±0.8 mm Hg (n=10, p<0.005, Student's t test. In comparison, addition of the same volume of saline at the same osmolality produced no significant change in IOP (−0.3±1.2 mmHg, n=6, p>0.8) 14 min later. Thus, in agreement with previous observations, the A₃AR agonist IB-MECA produced a rapid increase in IOP of 4.6±1.6 mmHg (n=6, p<0.05 by Student's t test) at 140 nM. At a 10-fold lower concentration, IB-MECA increased IOP by 2.2±0.5 mm Hg (p<0.02). In contrast, at a very high droplet concentration (1400 nM), IB-MECA exerted no significant effect (−1.2±1.9 mmHg), presumably because of cross-reaction with A_{2A}ARs.

To estimate an approximate range, “penetrance” was defined by Yang et al. as the ratio of the published Ki value at receptors in vitro to the minimally effective droplet concentration, for a number of adenosine agonists and antagonists. Thus, penetrance ranges from 1:100 to 1:1000 for purinergic drugs that have been tested in the mouse, and it is not very different from the drug penetrance of 1:100 for agents applied topically to rabbits and primates. This rule of thumb also applies to acylguanidine blockers and bumetanide, whose topical effects have also been studied in the mouse eye. Thus, the approximately 1:1000 penetrance that Yang et al. reported in the 2003 citation for MRS 1292 in the present experiments is consistent with past studies

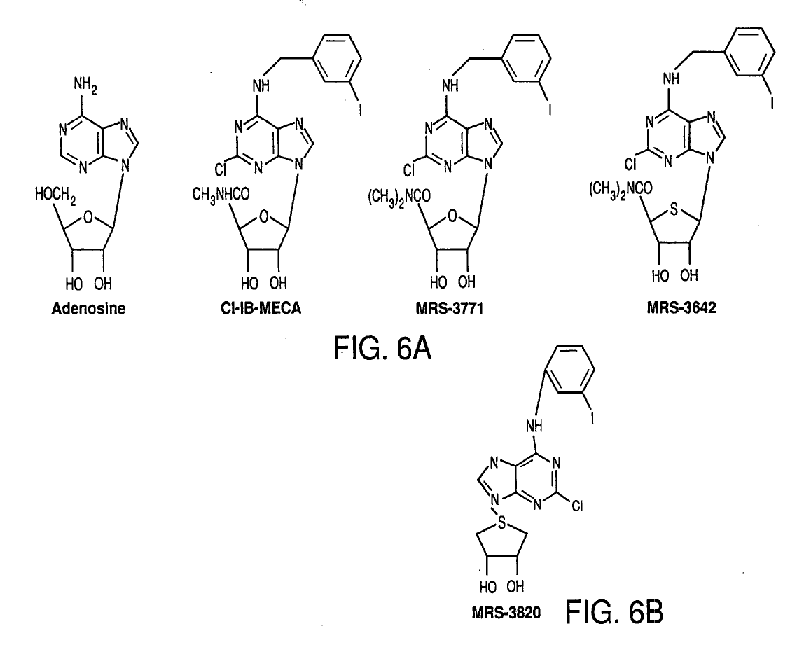

Using a similar strategy, two new A₃ antagonists, MRS-3642 and MRS-3771, were developed. The earlier compound, MRS-1292, was a modification of the A₃ agonist IB-MECA. The two new drugs (MRS-3642 and MRS-3771) were modifications of the more selective A₃ agonist Cl-IB-MECA, (structure shown in FIG. 6), and were therefore, anticipated to be even more selective than MRS-1292. Using the invasive servo-null technique, both new drugs, MRS-3642 and MRS-3771, were shown to be effective in lowering mouse IOP using invasive measurements (Table 1).

In Table 1, an endpoint of 10 minutes was used for the invasive measurements in light of the above-discussed downward drift of the IOP, even under control conditions. Also the need for smaller, more frequent droplets are discussed above with regard to the non-invasive technique using the pneumotonometer. As above, with the non-invasive protocol, the IOP was stable for 30 minutes under control conditions.

TABLE 1
IOP Effects of A₃ Agonists and Antagonists after Topical Application

| | | | | Pneumotonometer | | | Servo-Null | | |
|---|---|---|---|---|---|---|---|---|---|
| Class | Drug | Conc | | N | Δ(IOP) | P | N | Δ(IOP) | P |
| Nonselective agonist | Adenosine | 10 | mM | 9 | +4.8 ± 1.7 | <0.05 | 9 | +22.4 ± 6.5 | <0.01 |
| A₃ antagonist | MRS-1191 | 2.5 | mM (2% DMSO) | 9 | −3.9 ± 1.0 | <0.01 | 6 | −6.1 ± 1.1 | <0.001 |
| A₃ antagonist | MRS-3771 | 2.5 | mM (2% DMSO) | 4 | +0.3 ± 2.6 | >0.9 | | | |
| | | 250 | μM (2% DMSO) | 9 | +0.4 ± 0.6 | >0.5 | 9 | −3.0 ± 1.1 | <0.05 |
| A₃ antagonist | MRS-3642 | 250 | μM (2% DMSO) | 6 | +0.2 ± 1.1 | >0.8 | 10 | −4.2 ± 1.2 | <0.01 |
| A₃ agonist | CL-IB-MECA | 200 | nM (1% DMSO) | 6 | +0.7 ± 1.4 | >0.6 | 9 | +10.0 ± 2.9 | <0.01 |
| mono-propionyl CL-IB-MECA | MRS-3824 | 200 | nM (1% DMSO) | | | | 9 | +1.9 ± 1.3 | >0.1 |
| di-propionyl CL-IB-MECA | MRS-3823 | 200 | nM (1% DMSO) | 4 | +0.2 ± 0.8 | >0.8 | | | |
| di-acetyl ester of MRS-3642 | MRS-3826 | 250 | μM (2% DMSO) | 6 | −0.5 ± 0.8 | >0.5 | 4 | −4.0 ± 0.8 | <0.05 |
| mono-acetyl ester of MRS-3642 | MRS-3827 | 250 | μM (2% DMSO) | 9 | −0.8 ± 1.1 | >0.4 | 6 | −4.4 ± 1.3 | <0.05 |
| | | | | | | | 6 | −3.8 ± 0.8 | <0.01 |
| di-benzyl ester of MRS-3771 | MRS-3833 | 2 | μM (1% DMSO) | 6 | −0.2 ± 0.9 | >0.8 | | | |
| | | 200 | nM (1% DMSO) | 9 | −1.7 ± 0.6 | <0.03 | | 0.02 ± 3.8 | >0.9 |
| modified from MRS-3642 | MRS-3820 | 250 | μM (2% DMSO) | 6 | −4.2 ± 0.7 | <0.002 | 7 | −1.5 ± 0.6 | <0.05 |
| (rat/human A₃ antagonist) | | 75 | μM (0.6% DMSO) | | | | 6 | −3.1 ± 1.4 | >0.07 |
| | | 25 | μM (0.2% DMSO) | 6 | −0.6 ± 1.5 | >0.7 | 6 | −4.9 ± 1.7 | <0.05 |
| | | 5 | μM (0.2% DMSO) | | | | 6 | −2.2 ± 0.7 | <0.03 |
| Control | 2% DMSO | | | 9 | −1.4 ± 1.5 | >0.3 | 6 | −0.5 ± 0.6 | >0.4 |

Measured invasively, the mean±SEM reductions in IOP by MRS-3642 and -3771 were 4.2±1.2 mm Hg (N=10, P<0.01) and 3.0±1.0 mm Hg (N=10, P<0.03), respectively (Table 1). All data were obtained with Black Swiss mice. Topical addition of MRS-3771 also lowered IOP of C57 mice (by 3.3±0.6 mm Hg, N=6, P<0.01, Table 2). In contrast, MRS-3642 and MRS-3771 exerted no effect on IOP over the period of non-invasive measurements.

### Example 3

### Species Independent Effect of MRS-3820

In light of the foregoing cross-species results, a series of modifications of the two nucleoside-based A₃AR antagonists, MRS-3642 and MRS-3771, were developed to retain their cross-species effectiveness as A₃AR antagonists, and yet also to be sufficiently permeable across the cornea to produce rapid reductions in mouse IOP. This permitted a determination of each compound's efficacy by invasive measurements of IOP, and its ability to cross the cornea rapidly could be monitored by non-invasive measurements of IOP. A number of esters of MRS-3771 and 3642 were tested. Measured invasively, MRS-3824 was ineffective (Table 1). MRS-3833 reduced IOP slightly at 200 nM non-invasively, but was otherwise ineffective invasively and non-invasively (Table 1). MRS-3826 and -3827 lowered mouse IOP when measured invasively, but had no effect over the period of non-invasive measurement.

The nucleoside-based A₃AR antagonist, MRS-3820, was found to be effective, both by invasive and non-invasive measurement. MRS-3 820 (LJ-1251), a modification of MRS-3642, was prepared by L. S. Jeong for NIH. The structure of MRS-3820 (2-(2-chloro-6-(3-iodobenzylamino)-9H-purin-9-yl)tetrahydrothiophene-3,4-diol is shown in FIG. 6B. Notably, MRS-3820 was shown to lower non-invasively measured IOP within 20 minutes. As illustrated in Table 1, the concentration-response relationship was measured by both invasive and non-invasive techniques, although the magnitudes of the responses are not directly comparable because, as noted above, the protocols and time endpoints used with the two techniques are necessarily different. After topical application of 250 μM MRS-3820, the maximal response measured non-invasively was −4.2±0.7 mm Hg 30 min later (Table 1). Furthermore, the 250 μM MRS-3820 significantly reduced IOP after an even briefer interval, 20 min following application, by 3.4±0.7 mm Hg (N=6, P=0.004). The reduction in IOP produced by MRS-3820, measured both invasively and non-invasively indicates that this compound can rapidly penetrate the cornea to act as antagonist at A₃ receptors at the target site, the non-pigmented ciliary epithelial cells. In vitro work with ciliary epithelial cells and tissues cited above (Carre et al., supra, 1997; Mitchell et al., supra, 1999; Carre et al., supra, 2000) indicates that antagonism of the A₃ receptors reduces Cl⁻ channel activity of the non-pigmented ciliary epithelial cells. The ensuing reduced rate of aqueous humor formation reduced IOP.

TABLE 2:
Effect of topical MRS-3771 on C57 mouse intraocular pressure,
as measured invasively by the servo-null technique.

| | | BASELINE | MRS3771 | Change in |
|---|---|---|---|---|
| Exp. No. | Mouse Strain | (mm Hg) | (mm Hg) | IOP (mm Hg) |
| 050729B | C57 | 13.6 | 12.7 | −0.9 |
| 050729C | C57 | 16.4 | 11.8 | −4.6 |
| 050801C | C57 | 11.4 | 8.9 | −2.5 |
| 050801D | C57 | 22.8 | 19.4 | −3.4 |
| 050802A | C57 | 20.8 | 16.2 | −4.6 |
| 050802B | C57 | 14.3 | 10.5 | −3.9 |
| Mean ± SEM | | 16.5 ± 1.8 | 13.2 ± 1.6 | −3.3 ± 0.6 |
| Paired t-Test | | | | P = 0.002 |

The data of Table 1, taken together with unpublished binding measurements of MRS-3820 further verify that this compound functionally crosses species in binding to A₃ receptors. Experiments were performed using adherent CHO cells stably transfected with cDNA encoding the adenosine receptors (except for A_{2A}AR expressed in HEK 293 cells). Binding was carried out using [³H]CCPA, [³H]CGS-21680, and [¹²⁵I]AB-MECA as radioligands for A₁, A_{2A}, and A₃ receptors, respectively. Values presented herein are expressed as means±SEM, N=3-4. NECA was used to determine the non-specific binding. No significant difference was found between the binding of MRS-3820 to human and rat A₃ receptors. Specifically, the binding to human A₃ receptors was 4.2±0.5 nM and to rat A₃ receptors was 3.9±1.2 nM. The binding to A₃ receptors is also highly selective.

The potency (Ki, nM±SEM) at each of the four known human adenosine

receptors is: 2,485±940 nM (A₁), 341±74.6 nM (A_{2A}), <10% even at 10 μM (A_{2B}) and 4.16±0.50 nM (A₃). Furthermore, the binding of MRS-3820 functionally antagonizes the human A₃ receptors. In a cyclic AMP functional assay at the human A₃ receptor expressed in CHO cells, MRS-3820 dose-dependently shifted the agonist (Cl-IB-MECA) dose-response curve to the right as an antagonist, corresponding to a KB value of 1.92 nM. Thus, the effectiveness of MRS-3820 as a cross-species antagonist has been verified as a functional A₃ antagonist in human and rat (see, Jacobson and Gao, supra) and in mouse (Table 1). The large reduction in IOP of the normal mouse was an indication of the potential efficacy of the A₃AR-antagonists. The high selectivity of the drugs, as shown, reduced the possibility of side effects. In addition, the IOP effect provided evidence that MRS-3820 crossed the cornea.

Accordingly, the present invention provides a definitive method for delivering a species-independent, potent A₃ inhibitor across the corneal barrier to reduce activity of Cl⁻ channels of the non-pigmented ciliary epithelial (NPE) cells, thereby reducing the rate of aqueous humor formation and lowering intraocular pressure.

The disclosure of each patent, patent application and publication cited or described in this document is hereby incorporated herein by reference, in its entirety.

While the foregoing specification has been described with regard to certain preferred embodiments, and many details have been set forth for the purpose of illustration, it will be apparent to those skilled in the art without departing from the spirit and scope of the invention, that the invention may be subject to various modifications and additional embodiments, and that certain of the details described herein can be varied considerably without departing from the basic principles of the invention. Such modifications and additional embodiments are also intended to fall within the scope of the appended claims.

We claim:

1. A method for reducing intraocular pressure in an individual with an ocular disorder characterized by elevated intraocular pressure, the method comprising a step of administering to the individual an effective intraocular pressure-reducing amount of a cross-species pharmaceutical composition comprising an A₃ subtype adenosine receptor antagonist.

2. The method of claim 1, wherein the A₃ subtype receptor antagonist comprises a dihydropyridine, pyridine, pyridinium salt or triazoloquinazoline, or derivatives thereof expressly having A₃ subtype adenosine receptor antagonist activity.

3. The method of claim 1, wherein the A₃ subtype receptor antagonist in the method, comprises a composition consisting of MRS-1097, MRS-1191, MRS-1220, MRS-1523, MRS-1292, MRS-1523, MRS-3642, MRS-3771, MRS-3826, MRS-3827, MRS 1220, MRS-1649, LJ-1830, LJ-1831, LJ-1833, LJ-1834, LJ-1835, LJ-1836, LJ-1837 and MRS-3820.

4. The method of claim 3, wherein the A₃ subtype receptor antagonist comprises (2-(2-chloro-6-(3-iodobenzylamino)-9H-purin-9-yl)tetrahydrothiophene-3,4-diol or MRS-3820.

5. The method of claim 1, further comprising administering the pharmaceutical composition topically, systemically or orally.

6. The method of claim 5, further comprising administering pharmaceutical composition topically to the tear film of the patient's eye, in the form of an ointment, gel or eye drops.

7. The method of claim 6, further comprising impaling the cornea of the patient's eye with a microneedle or micropipette within 0.1-30 minutes of administering the composition to the tear film of the eye.

8. The method of claim 7, wherein the impaling step is part of invasively evaluating the efficacy of the composition for reducing the intraocular pressure of the eye.

9. The method of claim 8, comprising the invasive servo-null technique.

10. The method of claim 1, wherein elevated intraocular pressure is symptomatic of glaucoma in the patient.

11. A method for ensuring the delivery of a therapeutic composition for reducing intraocular pressure in an individual with an ocular disorder characterized by elevated intraocular pressure, the method comprising:
topically administering to a tear film of the individual's eye, an effective intraocular pressure-reducing amount of a pharmaceutical composition comprising an A₃ subtype adenosine receptor antagonist; and
impaling the cornea of the patient's eye with a microneedle or micropipette within 0.1-30 minutes of administering the composition to the tear film of the eye.

12. The method of claim 11, wherein the A₃ subtype receptor antagonist comprises a dihydropyridine, pyridine, pyridinium salt or triazoloquinazoline, or derivatives thereof expressly having A₃ subtype adenosine receptor antagonist activity.

13. The method of claim 11, wherein the A₃ subtype receptor antagonist in the method, comprises a composition consisting of MRS-1097, MRS-1191, MRS-1220, MRS-1523, MRS-1292, MRS-1523, MRS-3642, MRS-3771, MRS-3826, MRS-3827, MRS 1220, MRS-1649, LJ-1830, LJ-1831, LJ-1833, LJ-1834, LJ-1835, LJ-1836, LJ-1837 and MRS-3820.

14. The method of claim 13, wherein the A₃ subtype receptor antagonist comprises (2-(2-chloro-6-(3-iodobenzylamino)-9H-purin-9-yl)tetrahydrothiophene-3,4-diol or MS-3820.

15. The method for reducing intraocular pressure in an individual with an ocular disorder, comprising the step of administering to the individual an effective intraocular pressure-reducing amount of a cross-species A₃ subtype adenosine receptor antagonist prodrug which activates or enhances the production of an effective intraocular pressure-reducing amount of A₃ subtype adenosine in vivo for reducing intraocular pressure.
